(19) United States
(12) Patent Application Publication
(10) Pub. No.: US 2022/0233832 A1
(43) Pub. Date: Jul. 28, 2022

(54) Cryotherapy Systems and Methods

(71) Applicant: Arrinex, Inc., Redwood City, CA (US)

(72) Inventors: Brian Fahey, Menlo Park, CA (US); William Jason Fox, San Mateo, CA (US); Vahid Saadat, Atherton, CA (US)

(21) Appl. No.: 17/617,798

(22) PCT Filed: Jun. 12, 2020
(86) PCT No.: PCT/US2020/037497

§ 371 Date: Dec. 9, 2021

Related U.S. Application Data
(60) Provisional application No. 62/861,591, filed on Jun. 14, 2019.

(51) Int. Cl. A61M 31/00; A61B 18/02; A61M 5/32; A61M 19/00

(52) U.S. Cl. CPC A61M 31/00; A61B 18/02; A61M 5/3287; A61B 2018/0022; A61M 19/00; A61B 2018/0212; A61M 5/322

(57) ABSTRACT

In an example, a method for delivering an anesthetic agent to a target tissue in a nasal cavity of a patient includes inserting a cryotherapy device into the nasal cavity of the patient. The cryotherapy device includes a cryotherapy delivery feature and one or more protrusions. The method also includes positioning the cryotherapy delivery feature in contact with the target tissue, and delivering, using the cryotherapy delivery feature, a cryotherapy treatment to the target tissue. After inserting the cryotherapy device into the nasal cavity, the method includes actuating the one or more protrusions from a retracted state to an extended state. After actuating the one or more protrusions to the extended state, the method includes penetrating the target tissue with the one or more protrusions. After penetrating the target tissue, the method includes delivering, via the one or more protrusions, an anesthetic agent into the target tissue.

### CROSS-REFERENCE TO RELATED APPLICATIONS

The present application claims the benefit of priority to U.S. Provisional Pat. Appl. No. 62/861,591, filed on Jun. 14, 2019, the contents of which is hereby incorporated by reference in its entirety. The present applicant is also generally related to U.S. Pat. No. 9,687,288, filed Sep. 30, 2013, U.S. Pat. Publ. No. 2017/023147, filed Feb. 13, 2017, and U.S. Provisional Application No. 62/684,917, filed Jun. 14, 2018, the contents of which are hereby incorporated by reference in their entireties.

### FIELD

The present disclosure is related to the field of therapeutic thermal interventions and, more particularly, to apparatuses and methods for hypothermic thermal treatments (e.g., cryotherapies including hypothermic cooling and cryoablation).

### BACKGROUND

In general, thermal therapies involve treating tissue by inducing a temperature change that selectively induces alterations of the tissue, either temporarily or permanently. Depending on the tissue targeted for treatment, this thermal alteration may provide various benefits, including treatment of cardiac arrhythmia, destruction of cancerous tissue masses, and/or alteration of nerve signaling pathways. Ablation may be accomplished by applying heat (for example, with radiofrequency, laser, microwave, high intensity focused ultrasound (HIFU), or resistive heating methods) or by applying cooling energy (for example, using cryoablation techniques).

The term ‘cryotherapy’ describes a class of thermal therapies that involve inducing a relatively cold temperature in a tissue, and includes therapies generally referred to as therapeutic hypothermia and cryoablation. Depending on the temperatures and exposure times involved, the clinical goals of various cryotherapies may range from improved tissue healing/recovery (e.g., as with therapeutic hypothermia employed during physical therapy sessions) to selective tissue damage or destruction (e.g., during cryoablation used for neuromodulation or tumor-destruction purposes). Any tissue alteration introduced during cryotherapy may be temporary or permanent, depending on the tissue treated and one or more characteristics of the therapy applied to the tissue.

In practice, certain applications of cryotherapy may cause discomfort to a patient during and/or after treatment. For this reason, a medical practitioner may apply an anesthetic and/or an analgesic to the patient in tandem with the cryotherapy treatment. Indeed, medical practitioners generally endeavor to achieve adequate pain control when providing modern medical interventions. However, as some cryotherapies have moved away from operating room settings (where general anesthesia is available and practical) and into office-based settings (where only local anesthetics are generally available), existing pain control techniques for cryotherapy may be impractical or otherwise non-ideal for some procedures. Additionally, as medical practitioners have begun using cryotherapy interventions to target new anatomical regions, the potential physiological pathways and triggers for pain have also shifted, suggesting that improved solution pathways may emerge. Novel techniques, along with novel systems and apparatuses that enable these techniques, are required.

Rhinitis is defined as inflammation of the membranes lining the nose, and is characterized by nasal symptoms including itching, rhinorrhea, and/or nasal congestion. Chronic rhinitis affects millions of people and is a leading cause for patients to seek medical care. Medical treatment has been shown to have limited effects for chronic rhinitis sufferers and requires daily medication use or onerous allergy treatments, and up to 20% of patients may be refractory. Selectively interrupting the Posterior Nasal Nerves (PNN), Accessory Posterior Nasal Nerves (APNN), and/or other nervous structures in patients with chronic rhinitis (e.g., by applying cryotherapy within the nasal cavity to cryoablate these nerves) has been shown to improve symptoms with limited to elimination of side effects.

There are a number of possible physiological pathways by which the application of cryotherapy within the nasal cavity may lead to discomfort, either during the treatment or in the period following treatment. The mucosal and submucosal regions of the nasal cavity contain numerous sensory nerve fibers which primarily originate from the first and second branches (V1 and V2) of the trigeminal nerve (the fifth cranial nerve). Activation of these sensory nerves by cold stimuli may lead to sensations of pain. Pain may also be induced via an indirect activation of nerve endings, which may be possible via reflex arcs similar to the trigeminal-autonomic reflex (often associated with migraine, cluster headache, and other syndromes), and/or due to processes such as trigeminal sensitization, which may result in cold stimuli in the nose leading to discomfort felt in the anterior forehead, teeth/jaw, or in other regions. Additionally, the activation of reflex arcs leading to cold-stimulus headache (i.e. “ice-cream headache”) is possible in some scenarios, as the cooling of blood flowing through the treatment region, the cooling of nasally-inhaled air, and other mechanisms may all trigger significant cooling of regions that include the soft palate and the posterior pharyngeal wall. An ideal pain management solution would control for all pathways of possible discomfort associated with a particular intervention in order to ensure that a patient has a positive experience. In practice, solutions are needed that balance practicality, patient tolerance, and effectiveness.

The response of sensory afferent nerves to cold stimuli is complex. Within the nasal cavity, it is generally believed that there are at least two types of nerve fibers responsive to cold noxious stimuli (these fibers are oftentimes called ‘thermoreceptors’): (i) A-delta fibers, which are thick and myelinated, and (ii) C fibers, which are thin and unmyelinated. According to a scholarly publication by Wasner and colleagues (Wasner et al, Topical menthol—a human model for cold pain by activation and sensitization of C nociceptors, Brain 127:1159-1171, 2004—incorporated herein by reference), A-delta fibers are thought to carry painless cold sensations, whereas C fibers are thought to conduct pain. Again, according to Wasner and colleagues, studies suggest that cold-specific A-delta fibers may suppress the sensation of pain originating from C fibers, and accordingly the selective inhibition of A-delta fibers may amplify cold-induced pain. However, A-delta fibers are generally more sensitive than C fibers to topical anesthetics typically utilized in nasal procedures, such as lidocaine, tetracaine, and bupivacaine. As such, there may be a risk of inadequate anesthesia failing to inhibit pain-producing C fibers while at the same time exacerbating the situation by successfully inhibiting the A-delta fibers that help suppress these pain sensations. Accordingly, the most suitable approaches to anesthesia for cryotherapy applied within the nasal cavity will consider this complex response. Novel methods and enabling-devices would benefit this endeavor and improve care for patients

### SUMMARY

The present disclosure is related to systems, devices, and methods for applying anesthesia for thermal therapies. More specifically, the present disclosure relates to applying local anesthesia for hypothermic treatments of body tissues. This disclosure is particularly useful when treating patients during office-based procedures, or in other situations where general anesthesia is not available, practical, and/or advisable. The disclosure can be particularly useful during cryotherapy procedures applied within the upper airway.

It is an objective of the present disclosure to provide methods, devices, and systems that advance the delivery of local anesthetics with solutions that improve the balance between simplicity, practicality, and effectiveness. More specifically, it is an objective of the present disclosure to allow for adequate anesthesia for cryotherapies in the nasal cavity or other body lumens. Accomplishing this objective is valuable because it will improve the patient experience when receiving these valuable treatments which may encourage more patients to elect to receive said treatments.

In an example, an apparatus for delivering an anesthetic agent to a target tissue in a nasal cavity of a patient is described. The apparatus includes an elongated shaft and an applicator coupled to a distal end of the elongated shaft. The applicator includes a cryotherapy delivery feature configured to use a cryogen to apply thermal energy to the target tissue. The apparatus also includes one or more protrusions coupled to the applicator. Each protrusion includes a tip that is configured to penetrate the target tissue. Each protrusion is configured to actuated from a retracted state to an extended state. For each protrusion, (i) in the retracted state, the tip of the protrusion is at a first distance from an exterior surface of the applicator, (ii) in the extended state, the tip of the protrusion is at a second distance from the exterior surface of the applicator, and (iii) the second distance is greater than the first distance. The apparatus further includes one or more lumens extending through the elongated shaft. The one or more lumens are configured to transmit an anesthetic agent from an anesthetic agent to the one or more protrusions. Each protrusion includes an exit port that is configured to deliver the anesthetic agent to the target tissue in the nasal cavity.

In another example, an apparatus for delivering an anesthetic agent to a target tissue in a nasal cavity of a patient is described. The apparatus includes an elongated shaft and an applicator coupled to a distal end of the elongated shaft. The applicator includes a cryotherapy delivery feature configured to use a cryogen to apply thermal energy to the target tissue. The apparatus also includes a needle having a tip that is configured to penetrate the target tissue. The needle is actuatable between from a retracted state and an extended state. In the retracted state, the tip of the needle is at a first distance from the applicator. In the extended state, the tip of the needle is at a second distance from the applicator. The second distance is greater than the first distance. The apparatus further includes a needle guide system configured to facilitate translating the needle between the retracted state and the extended state.

In another example, a method for delivering an anesthetic agent to a target tissue in a nasal cavity of a patient is described. The method includes inserting a cryotherapy device into the nasal cavity of the patient. The cryotherapy device includes a cryotherapy delivery feature and one or more protrusions. The method also includes positioning the cryotherapy delivery feature in contact with the target tissue, and delivering, using the cryotherapy delivery feature, a cryotherapy treatment to the target tissue. After inserting the cryotherapy device into the nasal cavity, the method includes actuating the one or more protrusions from a retracted state to an extended state. After actuating the one or more protrusions to the extended state, the method includes penetrating the target tissue with the one or more protrusions. After penetrating the target tissue, the method includes delivering, via the one or more protrusions, an anesthetic agent into the target tissue.

The features, functions, and advantages that have been discussed can be achieved independently in various examples or may be combined in yet other examples further details of which can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 illustrates a simplified block diagram of a cryotherapy device, according to an example.

FIG. 2A illustrates a first side of a distal portion of the cryotherapy device of FIG. 1 positioned proximate to a target tissue, according to an example.

FIG. 2B illustrates a second side of the cryotherapy device of FIG. 2A, according to an example.

FIG. 3 illustrates a distal portion of the cryotherapy device of FIG. 1, according to another example.

FIG. 4A illustrates a distal portion of the cryotherapy device of FIG. 1 with a sheath in a distal position, according to another example.

FIG. 4B illustrates the cryotherapy device of FIG. 4A with the sheath in a proximal position, according to an example.

FIG. 5A illustrates a distal portion of the cryotherapy device of FIG. 1 in a covered state, according to another example.

FIG. 5B illustrates the cryotherapy device of FIG. 5A in a partially exposed state, according to an example.

FIG. 5C illustrates the cryotherapy device of FIG. 5A in a fully exposed state, according to an example.

FIG. 6 illustrates a distal portion of the cryotherapy device of FIG. 1, according to another example.

FIG. 7 illustrates a protrusion for delivering an anesthetic agent, according to an example.

FIG. 8 illustrates a distal portion of the cryotherapy device of FIG. 1, according to another example.

FIG. 9A illustrates a distal portion of the cryotherapy device of FIG. 1 with a needle in a retracted state, according to another example.

FIG. 9B illustrates the distal portion of the cryotherapy device of FIG. 9A with the needle in an extended state, according to an example.

FIG. 9C illustrates a cross-sectional view of the cryotherapy device of FIG. 9A, according to an example.

FIG. 10 illustrates a distal portion of the cryotherapy device of FIG. 1, according to another example.

FIG. 11A illustrates a distal portion of the cryotherapy device of FIG. 1 with a needle in a retracted state, according to another example.

FIG. 11B illustrates a cross-sectional view of the cryotherapy device of FIG. 11A, according to an example.

FIG. 11C illustrates the distal portion of the cryotherapy device of FIG. 11A with the needle in an extended state, according to an example.

FIG. 12 illustrates a flowchart for a method of using a cryotherapy device, according to an example.

FIG. 13 illustrates a flowchart for a method of using a cryotherapy device that can be used with the method shown in FIG. 12, according to an example.

FIG. 14 illustrates a flowchart for a method of using a cryotherapy device that can be used with the method shown in FIG. 13, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

The present disclosure is related to systems, devices, and methods for applying comfort control for thermal therapies. More specifically, the present disclosure relates to applying comfort control for hypothermic treatments of body tissues. The systems, devices, and methods of the present disclosure can be particularly useful when delivering thermal-based or other non-thermal treatments to patients in an office-based setting. Use of the disclosed methods, devices, and systems can improve management of pain during and/or following medical treatments.

Various aspects of the disclosure described herein may be applied to any of the particular applications set forth below or for any other types of thermal or non-thermal treatment systems or methods. The disclosure may be applied as a standalone system or method, or as part of an integrated medical treatment system.

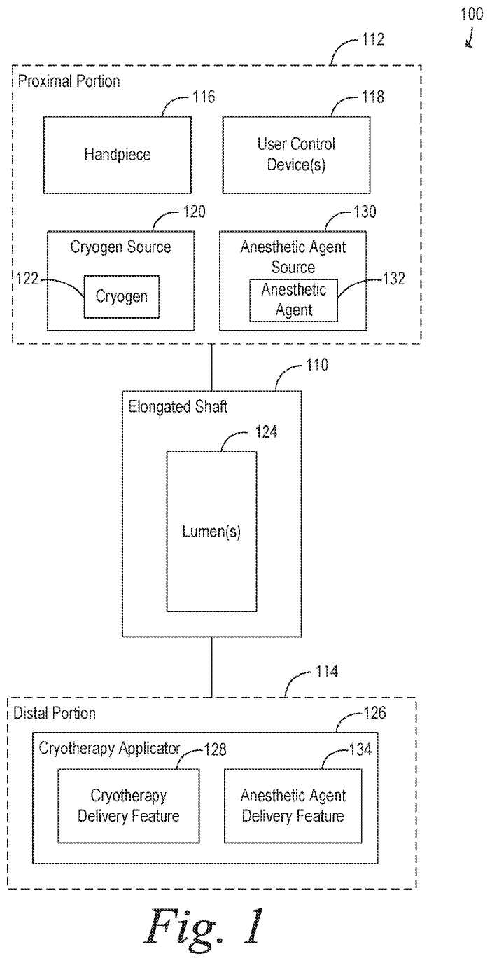

Referring now to FIG. 1, a simplified block diagram of a cryotherapy device 100 is shown according to an example. As shown in FIG. 1, the cryotherapy device 100 includes an elongated shaft 110 that extends between a proximal portion 112 of the cryotherapy device 100 and a distal portion 114 of the cryotherapy device 100. The elongated shaft 110 can be configured to be at least partially inserted in a nasal cavity of a patient. For example, the elongated shaft 110 can have a diameter between approximately 1 mm and approximately 4 mm. Additionally, for example, the elongated shaft 110 can be made from stainless Steel and/or semi-rigid polymer (e.g., such as Nylon or Pebax). The elongated shaft 110 can also be made of a combination of these.

Although the elongated shaft 110 is shown as being separate from the proximal portion 112 and the distal portion 114 in FIG. 1, the proximal portion 112 and/or the distal portion 114 of the cryotherapy device 100 can include respective portions of the elongated shaft 110. More generally, the proximal portion 112 can include one or more components of the cryotherapy device 100 that are located relatively farther away from a target tissue to be treated with cryotherapy during a cryotherapy procedure, and the distal portion 114 can include one or more components of the cryotherapy device 100 that are located relatively closer to the target tissue during the cryotherapy procedure. As used herein, the term “target tissue” means a tissue that is to be treated with cryotherapy during the cryotherapy procedure.

The proximal portion 112 can include a handpiece 116, one or more user control devices 118 (e.g., one or more knobs, one or more triggers, one-or more buttons, one or more switches, one or more levers, and/or one or more dials), a cryogen source 120 (e.g., a compressed gas canister and/or a fluid reservoir), and/or other features.

Within examples, the handpiece 116 can be configured to facilitate gripping and manipulating the cryotherapy device 100. For instance, the handpiece 116 can have a shape and/or a size that can facilitate a user performing a cryotherapy operation by manipulating the elongated shaft 110 and the distal portion 114 using a single hand. In one example, the handpiece 116 can have a shape and/or a size that facilitates the user holding the handpiece 116 in a pistol gripping manner (e.g., the handpiece 116 can have an axis that is transverse to an axis of the elongated shaft 110). In another example, the handpiece 116 can additionally or alternatively have a shape and/or a size that facilitates the user holding the handpiece 116 in a writing utensil gripping manner (e.g., the handpiece 116 can have an axis that is substantially parallel to an axis of the elongated shaft 110). Additionally or alternatively, the handpiece 116 can facilitate gripping and manipulating the cryotherapy device 100 by having a shape and/or a size that is greater than a shape and/or a size of the elongated shaft 110.

The cryogen source 120 can store a cryogen 122 such as, for example, nitrous oxide, liquid carbon dioxide, and/or liquid chlorofluorocarbon. In some implementations, the cryogen source 120 can be located in the handpiece 116. This can beneficially provide for a relatively compact size of the cryotherapy device 100 by, for example, reducing or eliminating relatively long external connections (e.g., tubes and/or cables) between the handpiece 116 and the cryogen source 120. In other implementations, the cryogen source 120 can be in a housing that is separate from the handpiece 116. This can beneficially allow the cryogen source 120 to store a relatively larger amount of the cryogen 122 without impairing the handling capabilities of the handpiece 116.

As shown in FIG. 1, the elongated shaft 110 can include one or more lumens 124. The lumen(s) 124 can include a first end that is coupled to the cryogen source 120 and a second end that is coupled to a applicator 126 at the distal portion 114 of the cryotherapy device 100. In this arrangement, the lumen(s) 124 can couple the cryogen source 120 and the applicator 126.

Within examples, the user control device(s) 118 can control a flow of the cryogen 122 from the cryogen source 120 to the applicator 126. For instance, the user control device(s) 118 can include one or more knobs, one or more triggers, one-or more buttons, one or more switches, one or more levers, and/or one or more dials that can be actuated to start, stop, increase, and/or decrease a flow of the cryogen 122 from the cryogen source 120 to the applicator 126. Also, within examples, the user control device(s) 118 can be located on the handpiece 116 and/or at a location that is separate from the handpiece 116.

As described above, the distal portion 114 includes the applicator 126. The applicator 126 includes a cryotherapy delivery feature 128. In general, the cryotherapy delivery feature 128 is configured to use the cryogen 122 to apply thermal energy to the target tissue. As such, the cryotherapy delivery feature 128 is coupled to the cryogen source 120 via the lumen(s) 124). In one example, the cryotherapy delivery feature 128 can include a balloon into which the cryogen 122 (e.g., in the form of a compressed liquid) can expand as a gas. As another example, the cryotherapy delivery feature 128 can include a metallic plate, which can be chilled through contact with the cryogen 122 (e.g., in the form of a circulating cooled fluid). In these examples, the cryotherapy delivery feature 128 includes an intermediary feature (e.g., the balloon and/or the metallic plate) that transfers the thermal energy from the cryogen 122 to the target tissue. This can beneficially help to improve the uniformity of the distribution of cold temperatures applied across a targeted region of tissue. This indirect application of cooling can also prevent cryogen substances (e.g. saline, or other liquids or gases) from direct exposure to the body in unwanted regions. For example, cold saline applied directly to the nasal cavity would run down a patient's throat, causing discomfort and possible tissue injury in unwanted regions.

In some implementations, the cryotherapy delivery feature 128 can have an active surface that is configured for contacting the target tissue such that relatively little or no thermal energy is applied to tissue regions remote from the active surface. For example, the cryotherapy delivery feature 128 can include the active surface and an inactive surface such that the cryotherapy delivery feature 128 applies the thermal energy to the target tissue contacting the active surface and does not apply the thermal energy to other tissue contacting the inactive surface. This can help to apply thermal energy in a relatively targeted manner to treat a specific target tissue.

In other implementations, an entirety of the cryotherapy delivery feature 128 can be active such that the cryotherapy delivery feature 128 applies the thermal energy omni-directionally. This can help to apply the thermal energy more broadly and, in some instances, can help to reduce a time for performing a cryotherapy procedure.

Accordingly, in this arrangement, the cryotherapy device 100 can be used to perform a cryotherapy procedure on the target tissue in the nasal cavity. For example, in operation, the cryotherapy device 100 can be inserted in the nasal cavity to position the applicator 126 at the target tissue (e.g., with the active surface(s) of the cryotherapy delivery feature 128 contacting the target tissue). After the applicator 126 is positioned at the target tissue, the user control device(s) 118 can be operated to cause the cryogen source 120 to supply the cryogen 122 to the cryotherapy delivery feature 128 via the lumen(s) 124 extending through the elongated shaft 110. The cryotherapy delivery feature 128 of the applicator 126 can use the cryogen 122 to apply the thermal energy to the target tissue to alter the target tissue and treat one or more conditions.

Within examples, the cryotherapy device 100 can additionally apply an anesthetic agent to the target tissue before, during, or after applying the thermal energy to the target tissue. For instance, as shown in FIG. 1, the cryotherapy device 100 can further include an anesthetic agent source 130 at the proximal portion 112. The anesthetic agent source 130 can store an anesthetic agent 132. As examples, the anesthetic agent 132 can include formulations of lidocaine, marcaine, tetracaine, bupivacaine, cocaine, and/or other anesthetic agents commonly utilized during medical procedures.

The anesthetic agent source 130 is coupled to the applicator 126 via the lumen(s) 124 extending through the elongated shaft 110. The applicator 126 can include an anesthetic agent delivery feature 134 that is configured to deliver the anesthetic agent 132 to the target tissue. For example, the anesthetic agent delivery feature 134 can include one or more protrusions and/or one or more needles that are configured to pierce and penetrate the target tissue. Additionally, for example, the protrusion(s) and/or the needle(s) can include one or more ports that provide for egress of the anesthetic agent 132 from the applicator 126 to the target tissue.

In some examples, the anesthetic agent source 130 can be separate from the handpiece 116. For instance, in one implementation, the anesthetic agent source 130 can include a syringe that contains the anesthetic agent 132. In this implementation, the syringe can be coupled to an infusion port on the handpiece 116 and a plunger of the syringe can be actuated to supply the anesthetic agent 132 from the anesthetic agent source 130 to the anesthetic agent delivery feature 134 (e.g., via the lumen(s) 124) and from the anesthetic agent delivery feature 134 to the target tissue. Thus, in this implementation, the anesthetic agent source 130 can provide a fluid pressure for delivering the anesthetic agent 132 through the lumen(s) 124 and out the anesthetic agent delivery feature 134 to the target tissue.

In other examples, the anesthetic agent source 130 can be integrated with the handpiece 116 and/or actuated by the user control device(s) 118. For instance, in one implementation, the anesthetic agent source 130 can be a disposable reservoir or a resusable reservoir that housed in the handpiece 116. The anesthetic agent source 130 can also include one or more valves and/or one or more pumps that facilitate supplying the anesthetic agent 132 from the anesthetic agent source 130 to the anesthetic agent delivery feature 134 of the applicator 126. The valve(s) and/or the pump(s) can be operable by the user control device(s) 118 to start, stop, increase, and/or decrease a flow of the anesthetic agent 132 from the anesthetic agent source 130 to the anesthetic agent delivery feature 134 of the applicator 126.

Accordingly, in the arrangement shown in FIG. 1, the cryotherapy device 100 can apply a combination of cryotherapy and anesthesia to treat the target tissue. As described above, applying local anesthesia using the anesthetic agent 132 before, during, and/or after applying the thermal energy of the cryotherapy procedure can beneficially help to improve patient comfort and/or increase access to types of cryotherapy procedures in office-based settings, which may not have been previously available due to a lack of general anesthesia capabilities in such office-based settings.

FIGS. 2A-11C show a plurality of distal portions that can be implemented in connection with the cryotherapy device 100 shown in FIG. 1, according to examples of the present disclosure. In particular, FIGS. 2A-11C show various example implementations for the elongated shaft 110, the applicator 126, the cryotherapy delivery feature 128, and/or the anesthetic agent delivery feature 134 shown in FIG. 1. The examples shown in FIGS. 2A-11C will now be described.

Referring now to FIGS. 2A-2B, a distal portion 214 of a cryotherapy device 200 is shown according to an example. The cryotherapy device 200 is substantially similar or identical to the cryotherapy device 100 shown in FIG. 1. As shown in FIGS. 2A-2B, the cryotherapy device 200 includes an applicator 226 coupled to a distal end 236 of an elongated shaft 210. As described above, the elongated shaft 210 can include the one or more lumens (e.g., the lumen(s) 124) that fluidly couple the applicator 226 to a cryogen source (e.g., the cryogen source 120).

In FIG. 2A, the applicator 226 is positioned proximate to a target tissue 238 that is to be treated during a cryotherapy procedure using the cryotherapy device 200. The applicator 226 includes a first side 240, which is positioned facing and in contact with the target tissue 238. As explained in further detail below, the first side 240 can include an active surface and thus provide a treatment side for applying cryotherapy to the target tissue 238. The applicator 226 can also include a second side 242, which is opposite the first side 240. Within examples, the second side 242 can be an active surface or an inactive surface. FIG. 2A shows the second side 242 and FIG. 2B shows the first side 240.

As shown in FIG. 2B, the applicator 226 includes a containable expandable member 244 in communication with the cryogen source via the lumen(s). Within examples, the expandable member 244 can be actuated between a collapsed state and an expanded state. The expandable member 244 can be actuated from the collapsed state to the expanded state by supplying the cryogen from the cryogen source to the expandable member 244 (e.g., via the lumen(s) 124). Specifically, when the cryogen source is operated (e.g., via the user control device(s) 118) to supply the cryogen to the expandable member 244, the expandable member 244 can fill with the cryogen (e.g., a gas from an evaporating liquid cryogen such as nitrous oxide) and expand the expandable member 244 from the collapsed state to the expanded state. In an example, the expandable member 244 can be actuated from the expanded state to the collapsed state by operating the user control device(s) 118) to cause the cryogen source to cease supplying the cryogen to the expandable member 244.

The expandable member 244 can be configured to transfer thermal energy from the cryogen to the target tissue 238. As such, the cryotherapy delivery feature 128 described above can include the expandable member 244 in the example shown in FIGS. 2A-2B.

In the collapsed state, the expandable member 244 can have a first size and/or a first shape. In the expanded state, the expandable member 244 can have a second size and/or a second shape. The first size and/or the first shape of the expandable member 244 in the collapsed state can facilitate inserting the expandable member 244 to through the nasal cavity to the target tissue. Whereas, the second size and/or the second shape of the expandable member 244 can help to engage the expandable member 244 with the target tissue and/or retain the expandable member 244 in a relatively fixed position at the target tissue. Accordingly, within examples, the first size can be less than the second size. In some examples, the first shape can be the same as the second shape. In other examples, the first shape can be different than the second shape. In some implementations the second shape can be based, at least in part, on a type of tissue that the expandable member 244 is configured to engage at the target tissue.

As shown in FIG. 2B, the applicator 226 can also include a scaffolding 246, which can provide structural rigidity to support the expandable member 244 (e.g., the scaffolding 246 can have a stiffness that is greater than a stiffness of the expandable member 244). Increasing or enhancing the structural rigidity of the applicator 226 to support the expandable member 244 can assist in manipulating a position of the applicator 226 with relatively greater precision and control than may be achieved if the expandable member 244 omitted the scaffolding 246. This can be particularly beneficial when manipulating the position of the applicator 226 when the expandable member 244 is in the collapsed state (e.g., during insertion of the applicator 226 to the target tissue).

In FIG. 2B, the scaffolding 246 extends along a longitudinal axis between a proximal end 240A of the expandable member 244 and a distal end 240B of the expandable member 244. This can assist with structural support of the expandable member and also provide mechanical strength to the distal portion of the cryotherapy device 200 to enhance maneuverability of the cryotherapy device 200 within the nasal cavity. However, in another example, the scaffolding 246 can additionally or alternatively extend in one or more directions that are transverse to the longitudinal axis. This can assist with further structural support of the expandable member 244, which may limit unwanted folding of the expandable member 244 that could occur as it makes contact with tissues during navigation to the desired treatment location.

Also, in FIG. 2B, the scaffolding 246 can be located in an internal cavity of the expandable member 244. This can help with maintaining a soft atraumatic surface for the distal portion of the cryotherapy device 200 and also, in some examples, allow for the scaffolding 246 to help deliver the cryogen centrally into the expandable member 244. However, in other examples, the scaffolding 246 can be located on an exterior surface of the expandable member 244. This can help to assemble the cryotherapy device 200 during a manufacturing process.

As examples, the expandable member 244 can be made from one or more materials including latex, silicone, urethane, and/or nylon. Also, as examples, the scaffolding 246 can be made from one or more materials including stainless steel, nitinol, and/or copper. Within examples, the scaffolding 246 has a stiffness that is greater than the expandable member 244.

As shown in FIG. 2B, the applicator 226 also includes a plurality of protrusions 248, which can extend from the scaffolding 246 near a central portion 250 of the applicator 226. In general, the protrusions 248 may be configured to allow for one or more fluids, one or more gases, and/or other materials to travel along one or more internal lumens 252 of the protrusions 248. For instance, as examples, the protrusions 248 can include a plurality of needles, cannulas, or other conduits through which the fluid(s), the gas(es), and/or the other material(s) can flow.

The protrusions 248 can also be configured to penetrate a soft tissue at or proximate to an implantation site (i.e., the target tissue 238). For instance, the protrusions 248 can be constructed from a material having a column strength that allows the protrusions 248 to penetrate the target tissue 238 without buckling or otherwise becoming kinked or bent. As examples, the protrusions 248 can be made from stainless steel and/or nitinol. Also, for instance, the protrusions 248 can have a beveled end that tapers to a point (i.e., similar to the tip of a needle) to facilitate penetrating the soft tissue and/or one or more frozen regions of the soft tissue (e.g., in instances in which the protrusions 248 are applied to the target tissue 238 after applying cryotherapy to the target tissue 238). As examples, to facilitate penetrating the soft tissue, the protrusions 248 can extend approximately 1 millimeter (mm) to approximately 4 mm from an exterior surface of the applicator 226.

Also, within examples, the protrusions 248 can include one or more exit ports 254 such that the protrusions 248 can deliver the fluid(s), the gas(es), and/or the other material(s) into the target tissue 238, which the protrusions 248 penetrate, or into other nearby tissues (e.g., tissues that are not penetrated and which may not be contiguous with the penetrated tissues). In some examples, for each protrusion 248, the exit port(s) 254 can be located at a tip 256 of the protrusion 248. In other examples, for each protrusion 248, the exit port(s) 254 can be additionally or alternatively located along a body of the protrusion 248 at one or more positions between the exterior surface of the applicator 226 and the tip 256 of the protrusion 248. In one example, when a protrusion penetrates a tissue, a portion of the exit ports 254 can be located in the penetrated tissue, whereas another portion of the exit ports 254 can be outside of the penetrated tissue such that the anesthetic agent can drip onto other nearby tissues

In some examples, the protrusions 248 can be actuated from a retracted state to an extended state. In the retracted state, the tips of the protrusions 248 can be positioned adjacent to the exterior surface of the applicator 226 or recessed in a body of the applicator 226 (i.e., at a position inward of the exterior surface). This can assist in providing the applicator 226 with a relatively low profile shape and/or size, which can help to mitigate (or prevent) unwanted tissue damage and/or discomfort while inserting the applicator 226 to the target tissue 238.

In the extended state, the tips 256 of the protrusions 248 can project outwardly from the exterior surface of the applicator 226. This can facilitate the protrusions 248 penetrating the target tissue 238. Within examples, (i) the tips 256 of the protrusions 248 can be at a first distance from the exterior surface of the applicator 226 when the protrusions 248 are in the retracted state, (ii) the tips 256 of the protrusions 248 can be at a second distance from the exterior surface of the applicator 226 when the protrusions 248 are in the retracted state, and (iii) the second distance can be greater than the first distance.

In one implementation, the protrusions 248 can be initially retracted within a body of the applicator 226 when the protrusions 248 are in the retracted state, and the protrusions 248 can then be deployed by a user (e.g., by operating the user control device(s) 118 of the proximal portion 112 of the cryotherapy device 100) to project outwardly when the protrusions 248 are in the extended state. In some implementations, the protrusions 248 can project outwardly from the body of the applicator 226 (e.g., via a spring-based mechanism that is released by manipulating user control device(s) 118) with a force that is sufficient to penetrate the target tissue 238. For instance, actuating the protrusions 248 from the retracted state to the extended state can include piercing and penetrating the target tissue 238 in some examples.

In another implementation, in the retracted state, the protrusions 248 can be initially folded against the exterior surface such that the first side 240 (i.e., the treatment side) of the applicator 226 is substantially flat. In this implementation, the protrusions 248 can actuate from the retracted state to the extended state by rotating (e.g., via a hinge or a joint) outward away from the exterior surface of the applicator 226.

Within examples, the user control device(s) 118 shown in FIG. 1 can be operable to actuate the protrusions 248 between the retracted state and the extended state. As examples, the user control device(s) 118 can include one or more knobs, dials, toggles, buttons, and/or other features that control the mechanical relays (e.g. pulleys, springs, etc.) for deploying and retracting the protrusions 248 between the retracted state and the extended state.

In FIG. 2B, the protrusions 248 extend from the scaffolding 246 and the central portion 250 of the applicator 226. This can help to effectively deliver an anesthetic agent to the center portion of the treated area while minimizing the number of protrusions and tissue penetration sites.

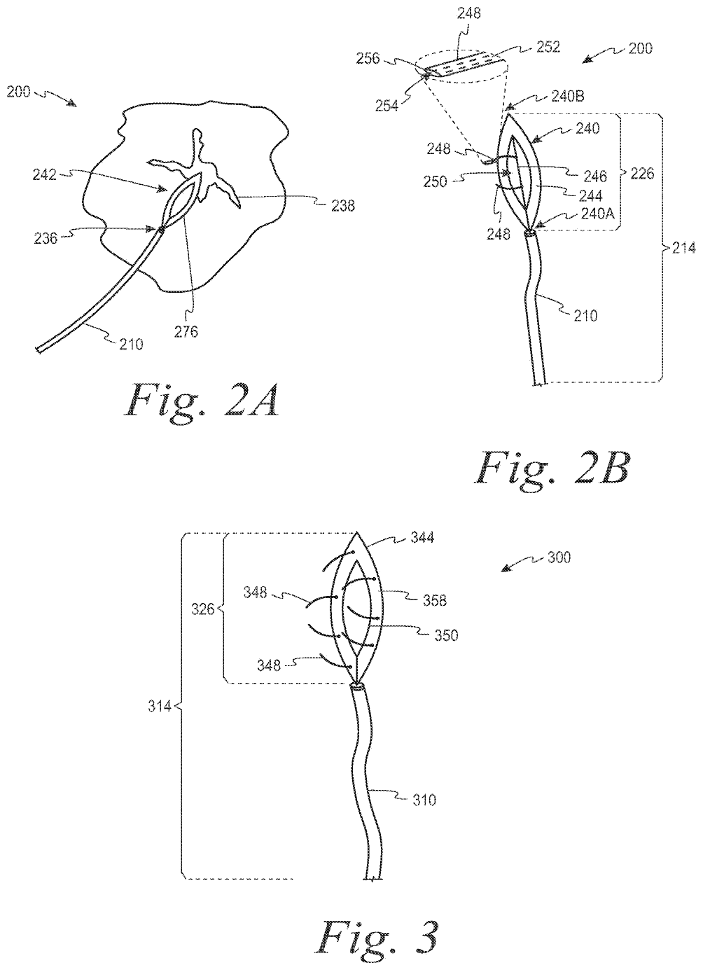

However, in other examples, the protrusions 248 can extend from other portions of the applicator 226. FIG. 3 shows a distal portion 314 of a cryotherapy device 300 according to another example. As shown in FIG. 3, the cryotherapy device 300 includes an applicator 326 coupled to an elongated shaft 310 and having an expandable member 344, as described above. The cryotherapy device 300 is substantially similar or identical to the cryotherapy devices 100, 200 described above, except the cryotherapy device 300 includes an applicator 326 having a plurality of protrusions 348 in an alternative arrangement. Specifically, in FIG. 3, the protrusions 348 extend from a peripheral portion 358 of the applicator 326, which extends around a central portion 350 of the applicator 326. Positioning the protrusions 348 at and/or around the peripheral portion 358 can help achieve broader delivery of an anesthetic agent, and therefore more complete coverage of a tissue region.

Although the protrusions 248 extend from the central portion 250 of the applicator 226 in FIG. 2 and the protrusions 348 extend from the peripheral portion 358 in FIG. 3, the protrusions 248, 348 can extend from the central portion 250 and the peripheral portion 358 in other examples.

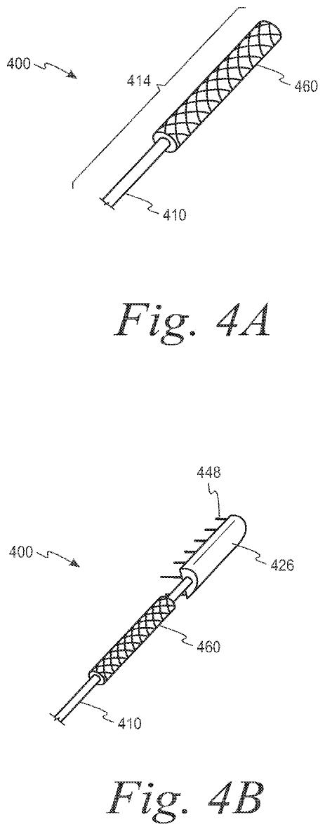

Referring now to FIGS. 4A-4B, a distal portion 414 of a cryotherapy device 400 is shown according to another example. As shown in FIGS. 4A-4B, the cryotherapy device 400 includes an applicator 426 coupled to an elongated shaft 410, as described above. The cryotherapy device 400 also includes a sheath 460 that is translatable along the elongated shaft 410 between (i) a distal position on the elongated shaft 410 shown in FIG. 4A and (ii) a proximal position on the elongated shaft 410 shown in FIG. 4B. In the distal position shown in FIG. 4A, the sheath 460 covers (or at least partially covers) the applicator 426 (i.e., the cryotherapy device 400 is in a covered state). Whereas, in the proximal position shown in FIG. 4B, the sheath 460 exposes the applicator 426 (i.e., the cryotherapy device 400 is in an exposed state).

As shown in FIG. 4B, the applicator 426 can include a plurality of protrusions 448 configured to deliver an anesthetic agent (e.g., the anesthetic agent 132) as described above. Within examples, the sheath 460 can actuate the protrusions 448 between the retracted state and the extended state described above. For instance, when the sheath 460 is in the distal position shown in FIG. 4A, the sheath 460 can apply a force that moves the protrusions 448 from an extended position (shown in FIG. 4B) to a retracted position in which the protrusions 448 are flattened against the applicator 426 and/or the protrusions 448 are recessed in the applicator 426. This can allow the applicator 426 to have a relatively slim and atraumatic profile, which can help facilitate inserting the applicator 426 in a nasal cavity to a target tissue (e.g., the target tissue 238 in FIG. 2).

Within examples, to actuate and/or maintain the protrusions 448 in the retracted state, the sheath 460 can circumferentially surround at least a portion of the applicator 426 adjacent to the protrusions 448. In some implementations, the sheath 460 can extend around an entire circumference of the applicator 426.

As described above, when the sheath 460 is in the proximal position exposing the applicator 426, the protrusions 448 extend outwardly from the applicator 426 toward the target tissue. Thus, when the sheath 460 is in the proximal position, the protrusions 448 can penetrate the target tissue and deliver the anesthetic agent to the target tissue. Within examples, after delivering the anesthetic agent to the target tissue, the sheath 460 can be actuated from the proximal position to the distal position to re-cover the applicator 426 and actuate the protrusions 448 from the extended state to the retracted state. Thus, positioning the sheath 460 in the distal position covering the protrusions 448 and the applicator 426 can additionally or alternatively facilitate withdrawing the applicator 426 out of the nasal cavity after completion of a procedure.

As examples, the sheath 460 can be comprised of a relatively soft and a relatively flexible material such as, for instance, nylon and/or another woven polymer. In other examples, the sheath 460 can be additionally or alternatively comprised of polytetrafluoroethylene (PTFE), a metallic braid, a metallic coiled ribbon, Polyimide, fluorinated ethylene propylene (FEP), and/or PEBAX.

In some examples, the sheath 460 can be similar to a hypotube adapted to slide along the elongated shaft 410 of the cryotherapy device 400. In some implementations, the cryotherapy device 400 can include a mechanical adjustment system that is operable by one or more user control devices (e.g., the user control device(s) 118 in FIG. 1) to adjust a position of the sheath 460 on the elongated shaft 410. For instance, in one implementation, a position of the sheath 460 can be adjusted by a user operating the one or more user control device(s) (e.g., such as a dial or toggle on the handpiece 116) that are in mechanical communication with a pulley or other mechanical system coupled to the sheath 460. In other implementations, the position of the sheath 460 can be adjusted directly by the user grabbing and moving the sheath 460 proximally and/or distally along the elongated shaft 410. Other examples are also possible.

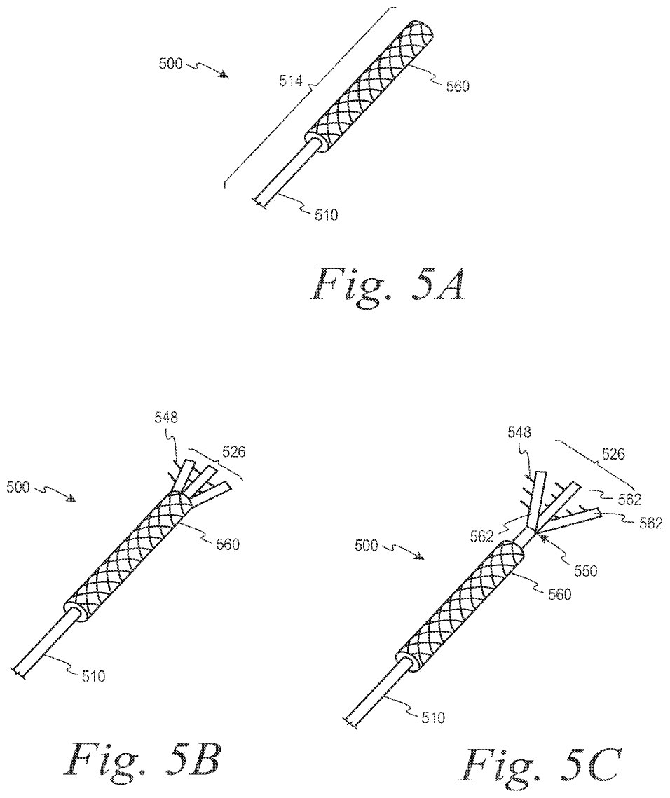

Referring now to FIGS. 5A-5C, a cryotherapy device 500 is shown according to another example. The cryotherapy device 500 is substantially similar or identical to the cryotherapy device 400 described above with respect to FIGS. 4A-4B, except the cryotherapy device 500 includes an applicator 526 that is configured differently than the applicator 426 shown in FIGS. 4A-4B.

FIG. 5A shows the cryotherapy device 500 in a covered state, FIG. 5B shows the cryotherapy device 500 in a partially exposed state, and FIG. 5C shows the cryotherapy device 500 in a fully exposed state. As shown in FIGS. 5A-5C, the cryotherapy device 500 includes the applicator 526 coupled to an elongated shaft 510, as described above. The cryotherapy device 500 further includes a sheath 560 that is translatable between (i) a distal position on the elongated shaft 510 shown in FIG. 5A, and (ii) a proximal position on the elongated shaft 510 shown in FIG. 5C. As described above, when the sheath 560 is in the distal position, the sheath 560 covers the applicator 526 and maintains a plurality of protrusions 548 in a retracted state. When the sheath 560 is in the proximal position, the sheath 560 exposes the applicator 526 and the protrusions 548 extend outwardly from the applicator 526 in an extended state.

In FIGS. 5A-5C, the applicator 526 includes a plurality of arms 562 extending from a central portion 550 of the applicator 526. In this example, the arms 562 include the protrusions 548. One or more of the arms 562 are movable about the central portion 550 such that the arms 562 can be collapsed to a position substantially aligned with a longitudinal axis of the sheath 560 and expanded to respective positions that diverge from the longitudinal axis of the sheath 560 (e.g., the arms 562 can fan out and/or spread out from each other). As an example, the arms 562 can be coupled to the central portion 550 by one or more hinges (e.g., one or more living hinges) and/or another pivoting structure.

In FIG. 5A, when the sheath 560 covers the applicator 526, the sheath 560 can cover the arms 562 and thereby position the arms 562 in the positions substantially aligned with the longitudinal axis of the sheath 560. As shown in FIG. 5B, as the sheath 560 moves from the distal position shown in FIG. 5A toward the proximal position shown in FIG. 5C, the sheath 560 initially exposes a portion of the applicator 526. This causes the arms 562 of the applicator 526 to begin spreading out from the central portion 550 (e.g., fan out and/or move radially away from each other). As shown in FIG. 5C, when the sheath 560 is at the proximal position, the arms 562 further spread out from the central portion 550 (i.e., the arms 564 expand radially outward from the central portion 550).

In this arrangement, the sheath 560 can actuate the arms 562 of the applicator 526 and the protrusions 548 to expand a size of the applicator 526 for delivering cryotherapy and/or the anesthetic agent, and reduce the size of the applicator 526 for inserting and/or withdrawing the applicator 526 in the nasal cavity. Expanding a size of the applicator 526 can allow for a larger treatment area while minimizing the profile of the cryotherapy device 500 during insertion and navigation of narrow aspects of the nasal cavity. This can facilitate reaching particular nerves for treatment.

In FIGS. 2A-5C, the applicator 226, 326, 426, 526 is shown with the protrusions 248, 348, 448, 548 of the applicator 226, 326, 426, 526 having a common length. However, in other examples, the protrusions 248, 348, 448, 548 in FIGS. 2A-5C can include a plurality of different lengths. Providing the protrusions 248, 348, 448, 548 with a plurality of different lengths can help to penetrate the target tissue 238 at a plurality of different depths and thus deliver the anesthetic agent 132 to the target tissue 238 at the different depths. This can help to achieve robust anesthetic coverage over a relatively broad tissue region.

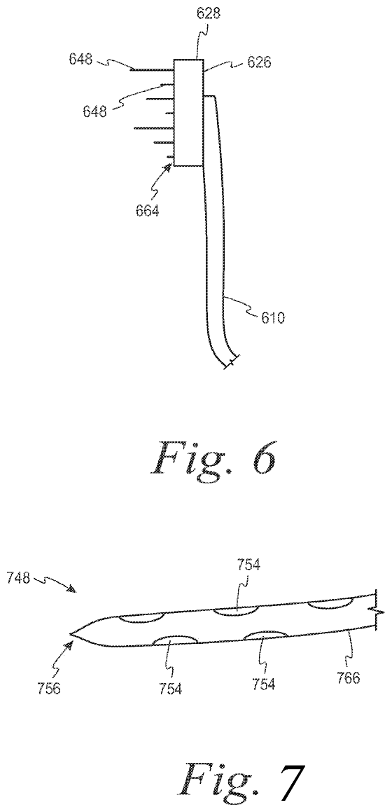

As one example, FIG. 6 shows a cryotherapy device 600 that includes an applicator 626 having a plurality of protrusions 648 of a plurality of different lengths (i.e., at least one of the protrusions 648 has a length that is different than a length of another one of the protrusions 648). As examples, the different lengths of the protrusions 648 can include approximately 1 mm, approximately 2 mm, and approximately 3 mm from an exterior surface 664 of the applicator 626. As shown in FIG. 6, the applicator 626 is coupled to an elongated shaft 610 and the applicator 626 can include a cryotherapy delivery feature 628, as described above. In some examples, a length of each of the protrusion(s) 648 can be different (i.e., each protrusion 648 can a respective length and all of the respective lengths can be different than each other).

As described above, the protrusions 248, 348, 448, 548, 648 can include one or more exit ports 254 that can facilitate delivering the anesthetic agent 132 from the cryotherapy device 100, 200, 300, 400, 500, 600 to the target tissue 238. FIG. 2B shows an example in which the exit port(s) 254 are located at the tips 256 of the protrusions 248. However, as described above, the exit port(s) 254 can additionally or alternatively be located on a body of each protrusion 248, 348, 448, 548, 648 at one or more positions between the exterior surface of the applicator 226, 326, 426, 526, 626 and the tip 256 of the protrusion 248, 348, 448, 548, 648.

As an example, FIG. 7 shows a protrusion 748 that includes one or more exit ports 754 along a body 766 of the protrusion 748. In the example shown in FIG. 7, the exit ports 754 are arranged in a relatively uniform pattern along and around a circumference of the body 766 of the protrusion 748. This can help to deliver the anesthetic agent 132 in a relatively uniform manner to the target tissue surrounding the protrusion 748. However, in other examples, the exit ports 754 can be arranged in other patterns. Within examples, the pattern of the exit ports 754 can be based on one or more criteria including, for instance, a type tissue of the target tissue 238, a size of the target tissue 238, a shape of the target tissue 238, a quantity of the protrusions 248, 348, 448, 548, 648, 748, and a spacing between the protrusions 248, 348, 448, 548, 648, 748.

In some examples, the cryotherapy devices 100, 200, 300, 400, 500, 600 described herein can be configured to adjust and/or control a temperature of the protrusions 248, 348, 448, 548, 648, 748 of the cryotherapy device 100, 200, 300, 400, 500, 600, 700. Specifically, the cryotherapy devices 100, 200, 300, 400, 500, 600 can be operable to warm the protrusions 248, 348, 448, 548, 648, 748 (i.e., apply heat to the protrusions 248, 348, 448, 548, 648, 748 to increase the temperature of the protrusions 248, 348, 448, 548, 648, 748).

Increasing the temperature of protrusions 248, 348, 448, 548, 648, 748 can provide a number of benefits. For example, increasing the temperature of protrusions 248, 348, 448, 548, 648, 748 can facilitate the protrusions 248, 348, 448, 548, 648, 748 penetrating the target tissue 238 after applying cryotherapy to the target tissue 238 (which may be frozen as a result of the cryotherapy applied to the target tissue 238). Increasing the temperature of the protrusions 248, 348, 448, 548, 648, 748 can additionally or alternatively assist in retaining the protrusions 248, 348, 448, 548, 648, 748 in the target tissue during or after cryotherapy is applied to the target tissue 238.

Additionally or alternatively, it can be beneficial to actively transfer heat from the protrusions 248, 348, 448, 548, 648, 748 to the target tissue before, during, or following a treatment. For example, after the target tissue 238 has been frozen as a result of a cryoablation procedure, transferring heat from the protrusions 248, 348, 448, 548, 648, 748 to the target tissue 238 can improve patient comfort.

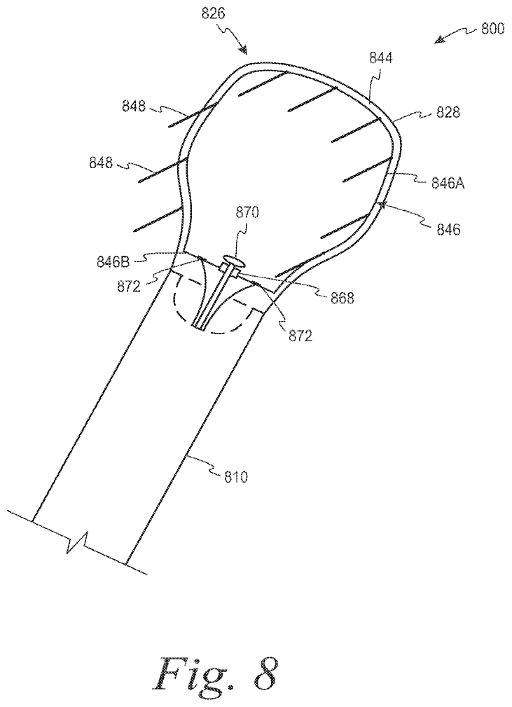

Referring now to FIG. 8, a cryotherapy device 800 that is configured to apply heat to a plurality of protrusions 848 is shown according to another example. As shown in FIG. 8, the cryotherapy device 800 includes an applicator 826 that is coupled to an elongated shaft 810, as described above. Accordingly, the elongated shaft 810 can include one or more lumens (e.g., the lumen(s) 124) that couple the applicator 826 to a cryogen source and/or an anesthetic agent source (e.g., the cryogen source 120 and/or the anesthetic agent source 130 in FIG. 1).

In FIG. 8, the applicator 826 includes a cryotherapy delivery feature 828 in the form of an expandable member 844 (e.g., a compliant/semi-compliant balloon). As examples, the expandable member 844 of the cryotherapy delivery feature 828 can be constructed of silicone, latex, and/or nylon. As described above, the expandable member 844 can be actuated from a collapsed state to an expanded state by supplying the cryogen from the cryogen source to the expandable member 844 (e.g., via the lumen(s) 124). In the expanded state, the expandable member 844 of the cryotherapy delivery feature 828 can use the cryogen to transfer a cold thermal energy to the target tissue 238 (i.e., apply cryotherapy to the target tissue 238).

Also, in FIG. 8, the applicator 826 includes a scaffolding 846, which can provide structural rigidity to support the expandable member 844. Increasing or enhancing the structural rigidity of the applicator 826 to support the expandable member 844 can assist in manipulating a position of the applicator 826 with relatively greater precision and control than may be achieved if the expandable member 844 omitted the scaffolding 846. Increasing or enhancing the structural rigidity can also help to maintain the expandable member 844 in a particular size and/or a particular shape when the expandable member 844 is in the collapsed state. This can, for example, help to position a plurality of protrusions 848 of the applicator 826 in an a particular arrangement for delivering the anesthetic agent 132 to the target tissue 238 while the expandable member 844 is in the collapsed state.

As shown in FIG. 8, the scaffolding 846 can be located in an internal cavity of the expandable member 844 and extend around a circumference of the expandable member 844 at or near outer peripheral edge of the expandable member 844. In this example, the scaffolding 846 is made from one or more materials that are electrically conductive and thermally conductive (hereinafter “electrically and thermally conductively material(s)”). For instance, the scaffolding 846 can include a material that is suitably resistive to generate a relatively high degree of heat in response to an electrical current applied to the scaffolding 846. As examples, the scaffolding 846 can be made from stainless steel and/or Nichrome.

In some examples, an entirety of the scaffolding 846 can include the electrically and thermally conductive material(s). In other examples, a first portion of the scaffolding 846 can be made from the electrically and thermally conductive material(s) and a second portion of the scaffolding 846 can be made from a material that is an electrical insulator and/or a thermal insulator (e.g., a plastic material). For instance, in FIG. 8, the scaffolding 846 can include a circumferential portion 846A that extends around the circumference and a central portion 846B that does not extend around the circumference. In this example, the circumferential portion 846A is made from the electrically and thermal conductive material(s) and the central portion 846B is made from a material that is an electrical insulator and/or a thermal insulator. As described further below, this can help to reduce (or minimize) generating heat at the central portion 846B of the scaffolding 846 at or near a location at which the cryogen 122 enters the expandable member 844.

In still other examples, the central portion 846B of the scaffolding 846 can be an electrically and thermally conductive material that is electrically isolated from the circumferential portion 846A. For example, the central portion 846B can be coupled to the circumferential portion 846A by one or more non-conductive joints or connectors 868, electrically isolating the central portion 846B of the scaffolding 846 from the circumferential portion 846A.

As shown in FIG. 8, the scaffolding 846 can also include one or more fenestrations 870 that allow the cryogen 122 (e.g., a compressed liquid cryogen that will expand into a gas at atmospheric pressure) to enter into the expandable member 844 and cool surrounding structures. As described above, the cryogen 122 can enter the cryotherapy device 800 through an intake port of the cryogen source 120 at or proximate to the handpiece 116 (see FIG. 1), and when a valve is shifted to be in an open position (e.g., responsive to operating the user control device(s) 118), the cryogen 122 can travel within the lumen(s) 124 of the elongated shaft 810 to reach the fenestration(s) 870. When the cryogen 122 exits the fenestration(s) 870, the cryogen 122 can expand into a gas within the expandable member 844.

Additionally, in FIG. 8, the applicator 826 can include a plurality of protrusions 848 coupled to the circumferential portion 846A of the scaffolding 846. As described above, each protrusion 848 can have a tip that is configured to penetrate the target tissue 238 and/or each protrusion 848 can be made from a material that sufficient column strength to puncture soft tissue without excessive bending or kinking. Additionally, as described above, the protrusions 848 can deliver the anesthetic agent 132 from then anesthetic agent source 130 to the target tissue 238 (e.g., via the lumen(s) 124 in the elongated shaft 110).

In some examples, the protrusions 848 are comprised of a thermally-conductive material, such as stainless steel. In some examples, the protrusions 848 are comprised of a material that is thermally conductive and has limited electrical conductivity such as, for example, diamond, glass, silicon, and/or ceramic. In other examples, the protrusions 848 can be comprised of a material that is both thermally conductive and electrically conductive, and the protrusions 848 can be coupled to the scaffolding 846 via one or more materials that are thermally-conductive with a high electrical resistivity, thereby substantially electrically-isolating the protrusions 848 from the scaffolding 846. This can assists in transmitting thermal energy from the protrusions 848 to the target tissue 238 while mitigating (or preventing) transmitting electrical energy from the protrusions 848 to the target tissue 238.

As described above, in some examples, the protrusions 848 can be actuated between the collapsed state and the extended state. In other examples, the protrusions 848 can substantially maintain a fixed position and/or a fixed orientation at all times. Although the protrusions 848 are coupled to the scaffolding 846 in FIG. 8, the protrusions 848 can be coupled to one or more other portions of the applicator 826 in other examples. For instance, as one example, one or more of the protrusions 848 can be coupled to the scaffolding 846 near a center of the expandable member 844 proximate to the fenestration 870.

The circumferential portion 846A of the scaffolding 846 is coupled to two electrical lead wires 872. In FIG. 8, the electrical lead wires 872 extend though at least one of the lumen(s) 124 in elongated shaft 810 to reach the handpiece (e.g., the handpiece 116 shown in FIG. 1). In some examples, the electrical lead wires 872 can be encased in an insulating material except for at the connection points found at each respective end of the electrical lead wires 873. In some examples, the electrical lead wires 872 can be coupled to the circumferential portion 846A of the scaffolding 846 in series or in parallel with a resistor, capacitor, or other electrical component.

Within or proximate to the handpiece, the electrical lead wires 872 can be coupled to opposite polarity terminals of an electrical power source (e.g., a battery and/or a power input module for adapting wall mains power). Additionally, within examples, the user control device(s) 118 shown in FIG. 1 can include an electrical switch coupled to the electrical lead wires 872 (e.g., in series with the power source), and actuatable between a first state and a second state. In the first state, the electrical switch is in an open position so that an electrical circuit is broken and no electrical current flows along the electrical lead wires 872 to the scaffolding 846. In the second state, the electrical switch completes the electrical circuit and causes an electrical current to flow from the power source through the electrical lead wires 872 to the circumferential portion 846A of the scaffolding 846. While current is flowing through the scaffolding 846, the scaffolding 846 transduces the electrical current into heat due to resistive heating (also known as Joule heating).

As the protrusions 848 and the scaffolding 846 are made from thermally conductive material(s), the protrusions 848 are thermally coupled to the scaffolding 846. Thus, responsive to the scaffolding 846 transducing the electrical current to the heat, the scaffolding 846 transmits the heat to the protrusions 848. Responsive to the heat received from the scaffolding 846, a temperature of the protrusions 848 increases. The protrusions 848 can further transmit the heat to the target tissue 238 as described above.

As described above, in implementations in which the central portion 846B is electrically isolated from the circumferential portion 846A of the scaffolding 846, the electrical current does not flow to the central portion 846B of the scaffolding 846. This can mitigate (or prevent) a risk of a short of the terminals of the electrical power source, reducing (or preventing) temperature elevations in the central portion of the delivery element. When desired, a user can operate the user control device(s) 118 to actuate the electrical switch from the second state back to the first state, thereby terminating the electrical current in the scaffolding 846 and cease generating the heat.

As described above, in some examples of the cryotherapy device 800 can allow for warm temperatures created from resistive heating of the circumferential portion 846A of the scaffolding 846 to reach the protrusions 848 while substantially keeping the protrusions 848 electrically isolated, thereby eliminating or substantially-eliminating any electrical current that could flow through the protrusions 848 into materials that the protrusions contact, for example into the patient's body.

Preferred examples will be configured such that the combination of material resistivity and power source voltage/current characteristics will result in mild temperature rises in the protrusions 848, for example temperature rises of less than 10° C. In some implementations, temperature rises in protrusions 848 can be equal to or less than approximately 1 degree Celsius to approximate 3 degrees Celsius. In general, the cryotherapy device 800 can be configured such that temperature rises induced by resistive heating do not melt or otherwise alter the expandable member 844 (or any other temperature sensitive element of the cryotherapy device 800).

Within examples, the cryotherapy device 800 can be operable to deliver the heat to the protrusions 848 before, during, and/or after insertion of the protrusions 848 into the target tissue 838. In some examples, the cryotherapy device 800 can warm the protrusions 848 only prior to inserting the protrusions 848 into the target tissue 838. In other examples, the cryotherapy device 800 can warm the protrusions 848 only after inserting the protrusions 848 in the target tissue 838. Warming the protrusions 848 can help to reduce a treatment time and improve a workflow for a physician.

Also, within examples, the user control device(s) 118 (including the electrical switch) can be operable to supply the electrical current from the electrical power source to the scaffolding 846 such that the scaffolding generates the heat continuously or intermittently in response to operation of the user control device(s) 118. For instance, in one example, the electrical power source can include a pulse width modulator that can convert a direct current power into a pulsed electrical signal. Other examples are also possible. In some implementations, providing continuous power can generate heat faster than pulsed power. However, pulsed power can provide a more gradual and more uniform temperature rise (and may additionally or alternatively provide for using a relatively lower-grade battery and/or drive system).

In some examples, the cryotherapy device 800 can include one or more sensors (e.g., one or more temperature sensors and/or one or more conductivity sensors) located the protrusions 848 or in another location on the cryotherapy device 800. In such examples, the sensor(s) can assist with determining whether or not the protrusions 848 have contacted and/or penetrated the target tissue 238, and/or whether warming should be enabled or disabled. For example, a conductivity sensor can measure the electrical impedance between different locations on a protrusion 848, or the electrical impedance between two or more protrusions 848, and compare the result to a threshold value that has been pre-programmed. Based on the comparison, the cryotherapy device 800 can determine that the protrusion 848 has penetrated tissue. This can help to mitigate a scenario where the protrusions 848 are warmed when the protrusions 848 are not in the desired or anticipated anatomical position, which could impact the efficacy of the procedure and/or have other unintended consequences.

As described above, the distal portion 914 is an implementation of the distal portion 114 of the cryotherapy device 100 shown in FIG. 1. In the examples described above, the protrusions 248, 348, 448, 548, 648, 848 generally extend from the applicator 226, 326, 426, 526, 626, 826 and the protrusions 248, 348, 448, 548, 648, 848 can be fluidly coupled to the anesthetic agent source 130 via at least one of the lumen(s) 124 in the elongated shaft 110 shown in FIG. 1. However, in other examples, the protrusions 248, 348, 448, 548, 648, 848 can be located at other locations of the cryotherapy device 100, 200, 300, 400, 500, 600, 800 and/or the protrusions 248, 348, 448, 548, 648, 848 can be coupled to the anesthetic agent source 130 by one or more conduits that are external to the elongated shaft 110.

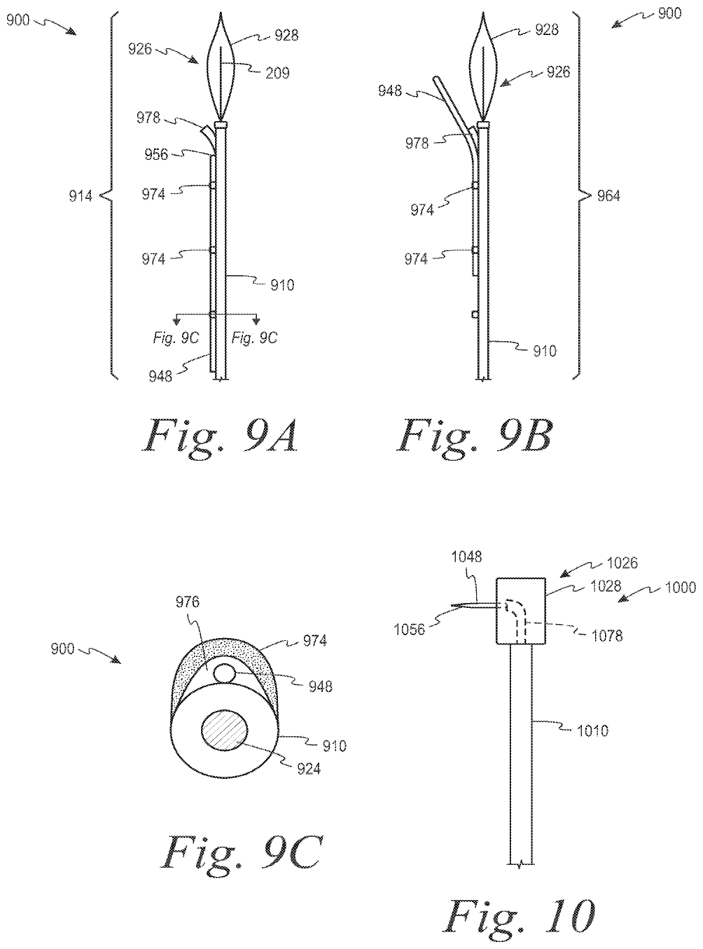

Referring now to FIGS. 9A-9C, a distal portion 914 of a cryotherapy device 900 is shown according to another example. In particular, FIG. 9A shows the cryotherapy device 900 with a needle 948 in a retracted state, FIG. 9B shows the cryotherapy device 900 with the needle in an extended state, and FIG. 9C shows a cross-sectional view of the cryotherapy device 900.

As shown in FIGS. 9A-9C, the cryotherapy device 900 includes an applicator 926 coupled to an elongated shaft 910, and the applicator 926 includes a cryotherapy delivery feature 928. As described above, the cryotherapy delivery feature 928 can be coupled to the cryogen source 120 via the lumen(s) 924 in the elongated shaft 910. Also, as described above, the cryotherapy delivery feature 928 can include an expandable member and/or an active surface (e.g., made from a metal) that can use the cryogen 122 to transfer cold thermal energy to the target tissue. In another example, the cryotherapy delivery feature 928 can include a thermoelectric device that is configured to generate cold thermal energy (e.g., via the Peltier effect or a similar process).

As shown in FIGS. 9A-9B, the cryotherapy device 900 also includes the needle 948, which can deliver the anesthetic agent 132 to the target tissue. For example, the needle 948 can include an internal lumen 924 (FIG. 9C) that extends between a tip 956 at a distal end of the needle 948 and the anesthetic agent source 130 at a proximal end of the needle 948. As described above, the tip 956 can be configured to pierce and penetrate the target tissue such that the needle 948 can deliver the anesthetic agent 132 to the target tissue. For instance, the tip 956 can have a shape that tapers inwardly to a point.

For instance, the needle 948 can have a gauge between 25 gauge and 30 gauge. Additionally, for instance, the needle 948 can be comprised of a material that retains sufficient column strength to puncture soft tissue without bending and/or kinking (e.g., nitinol or thin walled stainless steel tubes).

In FIGS. 9A-9B, the needle 948 extends along an exterior surface of the elongated shaft 910. More specifically, the needle 948 is translatable along the elongated shaft 910 between a proximal position shown in FIG. 9A and a distal position shown in FIG. 9B. As shown in FIG. 9A, the tip 956 of the needle 948 can be positioned proximal of a distal end 936 of the elongated shaft 910 in the proximal position (i.e., when the needle 948 is the retracted state). As shown in FIG. 9B, the tip 956 of the needle 948 can be positioned distal of the distal end 936 of the elongated shaft 910 in the distal position (i.e., when the needle 948 is the extended state).

More generally, (i) in the retracted state shown in FIG. 9A, the tip 956 of the needle 948 can be at a first distance from the applicator 926, (ii) in the extended state shown in FIG. 9B, the tip 956 of the needle 948 can be at a second distance from the applicator 926, and (iii) the second distance can be greater than the first distance.

In this arrangement, the needle 948 can be in (i) the retracted position while inserting the cryotherapy device 900 in a nasal cavity and withdrawing the cryotherapy device 900 from the nasal cavity, and (ii) in the extended position while penetrating and delivering the anesthetic agent 132 to the target tissue.

To facilitate translating the needle 948 between the retracted state and the extended state, the cryotherapy device 900 can include a needle guide system. For example, in FIGS. 9A-9B, the cryotherapy device 900 includes a plurality of guide hooks 974 along the exterior surface of the elongated shaft 910. In FIGS. 9A-9C, the guide hooks 974 are coupled to the elongated shaft 910 such that the guide hooks 974 and the exterior surface of the elongated shaft 910 define a plurality of apertures 976 that are axially aligned with each other. Although the apertures 976 are defined by a combination of the guide hooks 974 and the elongated shaft 910 in FIGS. 9A-9C, the apertures 976 can be defined by only the guide hooks 974 in other examples.

As shown in FIGS. 9A-9C, the needle 948 extends through one or more of the apertures 976 defined by the guide hooks 974 in the retracted state and the extended state. In this arrangement, the guide hooks 974 can assist in retaining the needle 948 in a plane that is substantially parallel to a plane in which the elongated shaft 910 is positioned when the needle 948 is in the retracted state. The guide hooks 974 can also assist in retaining the needle 948 against the elongated shaft 910 while translating the needle 948 from the proximal position shown in FIG. 9A to the distal position shown in FIG. 9B. Thus, the guide hooks 974 can define a first portion of a path for translating the needle 948 along an axis of extending through the apertures 976, which is substantially parallel to a longitudinal axis of the elongated shaft 910.

As shown in FIGS. 9A-9B, the needle guide system can also include a needle guide ramp 978 at or near the distal end of the elongated shaft 910. In general, the needle guide ramp 978 can be configured to guide the needle 948 along a second portion of the path that is transverse to the first portion of the path (i.e., defined by the axis extending through the apertures 976). In other words, the needle guide ramp 978 can be configured to bend the needle 948 as the needle 948 moves distally through the guide hooks 974 and along the elongated shaft 910 (i.e., diverting the tip 956 of the needle 948 from the axis of the apertures 976). This can beneficially help to direct the tip 956 of the needle 948 away from the elongated shaft 910 and/or towards the target tissue.

For example, as shown in FIGS. 9A-9B, a first end of the needle guide ramp 978 can located in-line with the axis of the apertures 976 and the needle guide ramp 978 can bend towards a second end of the needle guide ramp 978, which is located outward of the first end relative to the elongated shaft 910. In this arrangement, the needle guide ramp 978 can direct the needle 948 along a plane transverse to the plane of the elongated shaft 910. This can also help to deliver the anesthetic agent 132 to a portion of the target tissue that is adjacent to another portion of the target tissue to which the cryotherapy delivery feature 928 applies the cold thermal energy during cryotherapy.

As shown in FIG. 9A, the needle guide ramp 978 can extend over the tip 956 of the needle 948 when the needle 948 is at the proximal position. This can help to inhibit (or prevent) the needle 948 piercing and/or penetrating a tissue other than the target tissue while inserting the cryotherapy device 900 and/or while withdrawing the cryotherapy device 900 from the nasal cavity.

In FIG. 9C, the elongated shaft 910 has a circular cross-sectional shape. However, in another example, the exterior surface of elongated shaft 910 can include a hemi-cylindrical recess in which the needle 948 can be positioned. The hemi-cylindrical recess can allow for the needle 948 to translate along the elongated shaft 910 while reducing (or minimizing) a contribution of the needle 948 to an outer diameter of the cryotherapy device 900.

In some implementations, the needle 948 can be an integral component of the cryotherapy device 900 that can be moved along the elongated shaft 910. That is, the needle 948 can be coupled to the guide hooks 974 such that the needle 948 cannot be removed from the guide hooks 974. In other implementations, the needle 948 can be a separate component of a cryotherapy device 900 that can be repeatedly inserted into and/or removed from the guide hooks 974, and/or replaced by another needle 948. This can help to provide for the applicator 926 as a reusable component, and the needle 948 as a single-use component.

FIG. 10 shows a cryotherapy device 1000 according to another example. The cryotherapy device 1000 is substantially similar or identical to the cryotherapy device 900 of FIG. 9, except the cryotherapy device 900 includes a needle 1048 that is internal to an applicator 1026 and an elongated shaft 1010. The applicator 1026 is coupled to the elongated shaft 1010, and includes a cryotherapy delivery feature 1028 as described above. As shown in FIG. 10, the cryotherapy device 1000 also includes a needle guide ramp 1078 in an internal cavity of the applicator 1026. As described above, the needle guide ramp 1078 is configured to divert the needle 1048 from a first portion of a path along an axis (e.g., a longitudinal axis of the elongated shaft 1010) to a second portion of the path transverse to the axis. Specifically, in FIG. 10, the needle 1048 protrudes from the cryotherapy delivery feature 1028 at an angle that is substantially normal to a surface of the cryotherapy delivery feature 1028. This can help to apply the anesthetic agent 132 and the cool thermal energy to a common portion of the target tissue.

In FIG. 10, the needle 1048 is shown in an extended position. However, the needle 1048 can be translatable along the path defined by the needle guide ramp 1078 and the longitudinal axis of the elongated shaft 1010. In the retracted position, the needle 1048 can be positioned such that a tip 1056 of the needle 1048 is not exposed (i.e. the tip 1056 can be recessed within the applicator 1026).

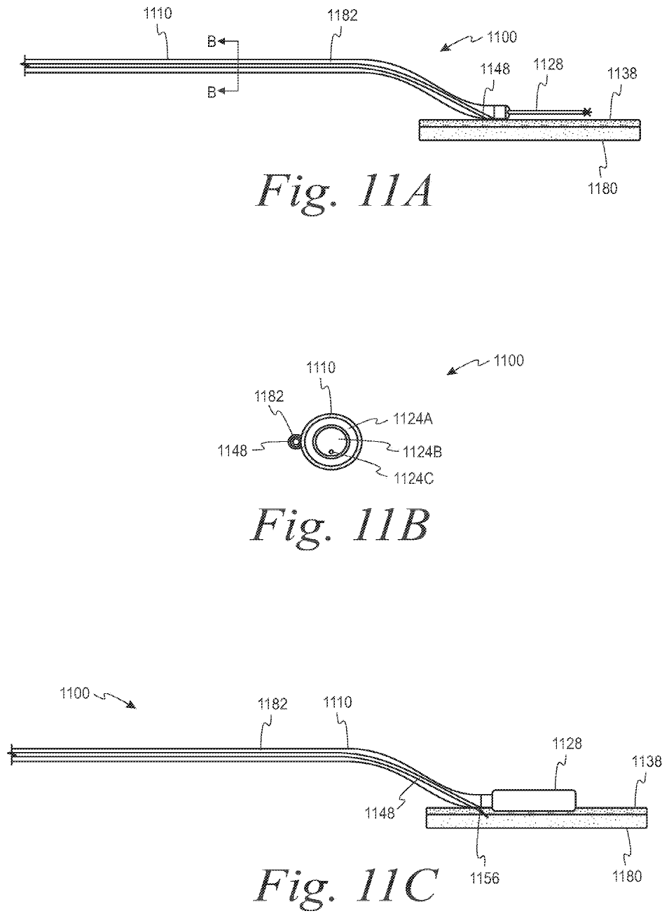

Referring now to FIGS. 11A-11C, a cryotherapy device 1100 is shown according to another example. Specifically, FIG. 11A shows a distal portion 1114 of the cryotherapy device 1100 (i) having a needle 1048 in a retracted state and (ii) in contact with a target tissue 1138 (e.g. upper airway mucosa/submucosa) overlying a semi-rigid structure 1180 (e.g. cartilage or bone). FIG. 11B shows a cross-sectional view of the cryotherapy device 1100 shown in FIG. 11A. FIG. 11C shows the distal portion 1114 of the cryotherapy device 1100 (i) having the needle 1048 in an extended state and (ii) in contact with the target tissue 1138 overlying the semi-rigid structure 1180.

The cryotherapy device 1100 includes a cryotherapy delivery feature 1128 that can deliver cryotherapy to the target tissue 1138. The cryotherapy device 1100 also includes an elongated shaft 1110 coupling the cryotherapy delivery feature 1128 to a handpiece (e.g., the handpiece 116). The elongated shaft 1110 is shaped so that the cryotherapy delivery feature 1128 is off-axis to a central axis of the cryotherapy device 1100 (i.e., a longitudinal axis of the elongated shaft 1110 is in a different plane than a longitudinal axis of the cryotherapy delivery feature 1128). To facilitate shaping the elongated shaft 1110, the elongated shaft 1110 can be (i) semi-flexible and pre-shaped by a manufacturer, (ii) constructed of malleable material so shape can be defined by the user, and/or (iii) comprised of a multi-flexible shaft with articulating features that allow the user to flex and bend the elongated shaft 1110 during delivery and adjust a shape while the elongated shaft 1110 is in the patient. For instance, the elongated shaft 1110 can be made from a material that can be manipulated to adjust a shape of the elongated shaft 1110 and then retain the shape after being manipulated.

As shown in FIGS. 11A-11C, the cryotherapy device 1100 can include a needle guide system to assist translating the needle 1048 between the retracted state and the extended state, as described above. For instance, in FIGS. 11A-11C, the cryotherapy device 1100 can include a needle conduit 1182 coupled to the elongated shaft 1110. The needle conduit 1182 can have a size and a shape that allows the needle 1048 to be positioned and translate in the needle conduit 1182. In one example, the needle 1148 can have a gauge between 25 gauge and 30 gauge (e.g., the needle 1148 can be a relatively thin-walled metallic tube). Also, as an example, the needle conduit 1182 can have a diameter that is between approximately 0.05 mm and approximately 0.15 mm larger than an outer diameter of the needle 1148. This can help ensure that the needle 1148 can translate through the needle conduit 1182 while still providing the needle 1148 with a diameter that can achieve sufficient column support to facilitate piercing and penetrating the target tissue 1138.

In an example, the needle conduit 1182 can be comprised of shapeable material such as, for instance, a heat shaped polymer. In one implementation, the needle conduit 1182 and a jacket of the elongated shaft 1110 can be made from a single extruded multi-lumen. A distal end of the needle conduit 1182 can be proximal of the cryotherapy delivery feature 1128 and a proximal end of the needle conduit 1182 can be at the handpiece (e.g., the handpiece 116).

As shown in FIG. 11B, the needle conduit 1182 and the needle 704 are aligned in the opposite plane of a bend in the elongated shaft 1110 (e.g., the needle 704 can be on top or on bottom of the elongated shaft 1110 and have a shape that corresponds to the bend of the elongated shaft 1110). The elongated shaft 1110 can include an outer lumen 1124A and an inner lumen 1124B, which is concentric with and inside of the outer lumen 1124A. In the inner lumen 1124B is a cryogen lumen 1124C that can deliver a cryogen (e.g., the cryogen 122) to the cryotherapy delivery feature 1128. In this example, an exhaust from the cryogen can return proximally through the inner lumen 1124B.

In FIG. 11B, the outer lumen 1124A and the inner lumen 1124B are separated by a gap. The gap can provide insulation along the elongated shaft 1110 to help reduce (or prevent) the elongated shaft 1110 and the needle conduit 1182 from decreasing in temperature to a temperature level that may ablate tissue outside of the target tissue 1138. The gap can also help inhibit (or prevent) an anesthetic agent (e.g., the anesthetic agent 132) in needle 1148 from freezing.

As noted above, FIG. 11A shows the needle 1148 in the retracted state and FIG. 11C shows the needle 1148 in the extended state. As shown in FIG. 11A, when the needle 1148 is in the retracted state, the needle 1148 can be completely retracted within the needle conduit 1182, allowing the cryotherapy delivery feature 1128 to make contact with the target tissue without piercing the target tissue 1138. As shown in FIG. 11C, when the needle 1148 is in the extended state, a tip 1156 of the needle 1148 is advanced more distally into the target tissue 1138.

In some implementations, the needle 1148 can be an integral component of the cryotherapy device 1100 that can be moved along the elongated shaft 1110. That is, the needle 1148 can be coupled to the needle conduit 1182 such that the needle 1148 cannot be removed from the needle conduit 1182. In other implementations, the needle 1148 can be a separate component of a cryotherapy device 1100 that can be repeatedly inserted into and/or removed from the needle conduit 1182, and/or replaced by another needle in the needle conduit 1182.

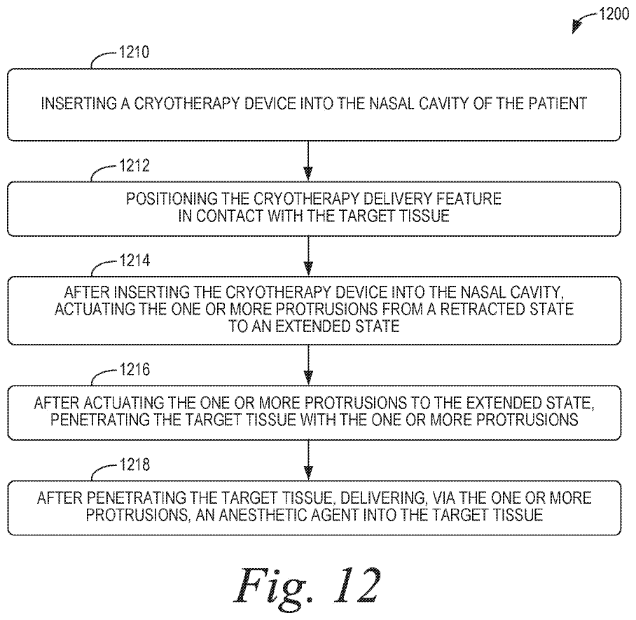

Referring now to FIG. 12, a flowchart of a method 1200 for delivering an anesthetic agent to a target tissue in a nasal cavity of a patient is shown according to an example. As shown in FIG. 12, the method 1200 can include inserting a cryotherapy device into the nasal cavity of the patient at block 1210. The cryotherapy device can include a cryotherapy delivery feature and one or more protrusions. At block 1212, the method 1200 can include positioning the cryotherapy delivery feature in contact with the target tissue. At block 1214, the method 1200 can include delivering, using the cryotherapy delivery feature, a cryotherapy treatment to the target tissue. After inserting the cryotherapy device into the nasal cavity at block 1210, the method 1200 can include actuating the one or more protrusions from a retracted state to an extended state at block 1216. After actuating the one or more protrusions to the extended state at block 1216, the method 1200 can include penetrating the target tissue with the one or more protrusions at block 1218. After penetrating the target tissue at block 1218, the method 1200 can include delivering, via the one or more protrusions, an anesthetic agent into the target tissue.

In some examples, penetrating the target tissue at block 1218 can be performed prior to delivering the cryotherapy treatment at block 1214. This can help to reduce a force for penetrating the target tissue as the target tissue can become more difficult to penetrate after delivering the cryotherapy to the target tissue. In some implementations, the protrusions can remain in the target tissue while delivering the cryotherapy and for at least a period of time after delivering the cryotherapy at block 1214. This can allow for a plurality of modes of operation (e.g., applying anesthesia prior to cryotherapy and then warming (with or without additional anesthesia after cryotherapy). In other examples, penetrating the target tissue at block 1218 can be performed only after delivering the cryotherapy at block 1214. This may be beneficial in instances in which the protrusions may interfere with cryotherapy. In some examples, the method 1200 can include delivering the cryotherapy treatment at block 1214 and delivering the anesthetic agent at block 1218 simultaneously.

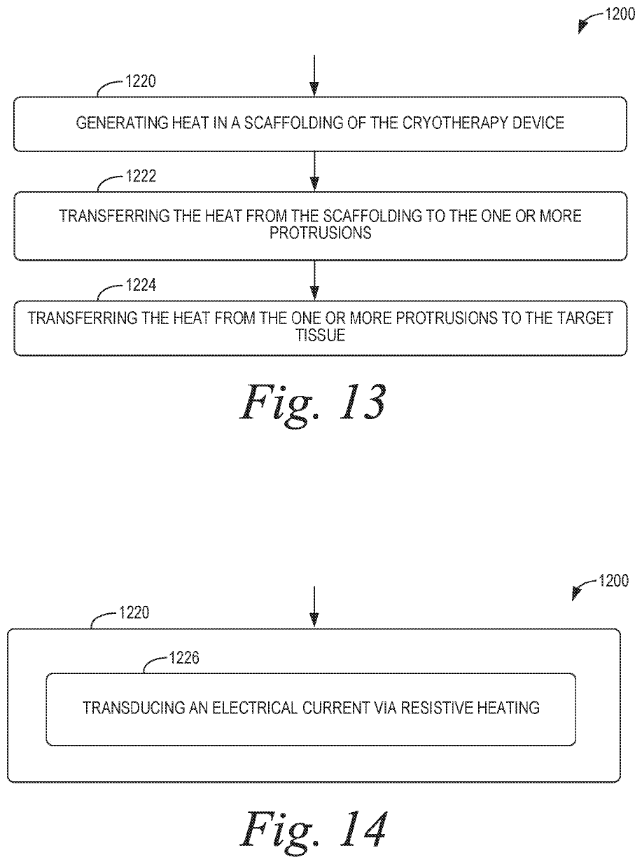

FIGS. 13-14 depict additional aspects of the method 1200 according to further examples. As shown in FIG. 13, the method 1200 can also include (i) generating heat in a scaffolding of the cryotherapy device at block 1220, (ii) transferring the heat from the scaffolding to the one or more protrusions at block 1222, and (iii) transferring the heat from the one or more protrusions to the target tissue at block 1224. As shown in FIG. 14, generating the heat in the scaffolding at block 1220 can include transducing an electrical current via resistive heating at block 1226.

In the examples described above, the method 1200 involves delivering the anesthetic agent to the target tissue via the protrusions. However, in other examples, the method 1200 can include delivering a non-pharmaceutical agent is delivered to the target tissue. As examples, the non-pharmaceutical agent can include water or saline.

In some examples of methods or devices described herein, the cryotherapy device can be adapted such that the insertion of protrusions such as needles into tissues can be gradual with time and/or performed at an adjustable length based upon a user action. For example, rotating a dial or otherwise manipulating a control feature on or proximate to a device handpiece can slowly expand or deploy a needle protrusion into tissue, allowing for precise control of penetration depth and thereby precise control over the depth and types of tissue impacted by the therapies delivered via the protrusions.

In some examples of the methods or devices described herein, the protrusions at the distal end of the device are adapted to provide cooling and in effect serve as the cryotherapy delivery element(s). In some examples, the protrusions can be adapted to deliver both cooling and heating energy based upon a user input or the user manipulating a control feature proximate to the handpiece of the device.

Though the disclosures presently-disclosed have primarily been discussed in the context of cryotherapy, the devices, systems, and methods described can have applicability with other ablative and non-ablative surgical techniques. For example, examples can include devices, systems, and methods that utilize heating/hyperthermia therapies. Examples utilizing heating/hyperthermia therapies can be similar in structure and steps as examples utilizing hypothermic therapies. Sources of heat for use with hyperthermia-based therapies can include RF energy, microwave energy, ultrasound energy, resistive heating, exothermic chemical reactions, combinations thereof and other heat sources known to those skilled in the art. Further, the disclosure can be applied as a standalone system or method, or as part of an integrated medical treatment system. It shall be understood that different aspects of the disclosure can be appreciated individually, collectively, or in combination with each other.

The methods described herein can be utilized effectively with any of the examples or variations of the devices and systems described above, as well as with other examples and variations not described explicitly in this document. The features of any of the systems or system components described in any of the examples herein can be used in any other suitable example of a system or system component.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

What is claimed is:

1. An apparatus for delivering an anesthetic agent to a target tissue in a nasal cavity of a patient, the apparatus comprising:
an elongated shaft;
an applicator coupled to a distal end of the elongated shaft, wherein the applicator comprises a cryotherapy delivery feature configured to use a cryogen to apply thermal energy to the target tissue;
one or more protrusions coupled to the applicator, wherein each protrusion comprises a tip that is configured to penetrate the target tissue, wherein each protrusion is configured to actuated from a retracted state to an extended state, wherein for each protrusion:
(i) in the retracted state, the tip of the protrusion is at a first distance from an exterior surface of the applicator,
(ii) in the extended state, the tip of the protrusion is at a second distance from the exterior surface of the applicator, and
(iii) the second distance is greater than the first distance; and
one or more lumens extending through the elongated shaft, wherein the one or more lumens are configured to transmit an anesthetic agent from an anesthetic agent to the one or more protrusions,
wherein each protrusion comprises an exit port that is configured to deliver the anesthetic agent to the target tissue in the nasal cavity.

2. The apparatus of claim 1, wherein the cryotherapy delivery feature comprises a balloon.

3. The apparatus of any one of claims 1-2, wherein, when the one or more protrusions are in the extended state, the one or more protrusions extend away from the exterior surface of the applicator by between about 1 mm to about 4 mm.

4. The apparatus of any one of claims 1-3, further comprising:
an scaffolding coupled to the cryotherapy delivery feature,
wherein the scaffolding has a stiffness that is greater than a stiffness of the cryotherapy delivery feature, and

5. The apparatus of any one of claims 1-4, wherein the one or more protrusions are coupled to the scaffolding.

6. The apparatus of any one of claims 1-5, wherein the one or more protrusions extend from a peripheral portion of the applicator.

7. The apparatus of any one of claims 1-6, wherein each protrusion is coupled to the applicator via a hinge such that the protrusion can actuate from the retracted state to the extended state by rotating, via the hinge, outward away from the exterior surface of the applicator.

8. The apparatus of any one of claims 1-7, wherein a length of a first protrusion of the one or more protrusions in the extended state is different than a length of a second protrusion of the one or more protrusions in the extended state.

9. The apparatus of any one of claims 1-8, wherein a length of each of the one or more protrusions is different.

10. The apparatus of any one of claims 1-9, further comprising:
a sheath that is translatable along the elongated shaft between (i) a distal position on the elongated shaft and (ii) a proximal position on the elongated shaft,
wherein, in the distal position, the sheath at least partially covers the applicator and retains the one or more protrusions in the retracted state, and
wherein, in the proximal position, the sheath exposes the applicator and allows the one or more protrusions to be in the extended state.

11. The apparatus of claim 10, wherein the applicator comprises a plurality of arms that expand radially outward from a central portion when the sheath is in the proximal position, and
wherein the plurality of arms comprise the one or more protrusions.

12. The apparatus of any one of claims 1-11, wherein, for each protrusion, the exit port comprises a plurality of exit ports located along a length of the protrusion.

13. The apparatus of any one of claims 1-12, further comprising:
a scaffolding extending around a circumference of the cryotherapy delivery feature, wherein the scaffolding comprises a material that is thermally conductive and electrically conductive; and
one or more electrical lead wires coupled to the scaffolding and configured to couple the scaffolding to an electrical power source,
wherein the scaffolding is configured to generate heat responsive to an electrical current from the electrical power source, and
wherein the scaffolding is thermally coupled to the one or more protrusions and configured to transfer heat from the scaffolding to the one or more protrusions.

14. An apparatus for delivering an anesthetic agent to a target tissue in a nasal cavity of a patient, the apparatus comprising:
an elongated shaft;
an applicator coupled to a distal end of the elongated shaft, wherein the applicator comprises a cryotherapy delivery feature configured to use a cryogen to apply thermal energy to the target tissue;
a needle comprising a tip that is configured to penetrate the target tissue, wherein the needle is actuatable between from a retracted state and an extended state, wherein:
(i) in the retracted state, the tip of the needle is at a first distance from the applicator,
(ii) in the extended state, the tip of the needle is at a second distance from the applicator, and
(iii) the second distance is greater than the first distance; and
a needle guide system configured to facilitate translating the needle between the retracted state and the extended state.

15. The apparatus of claim 14, wherein the needle guide system comprises:
a plurality of guide hooks coupled to the elongated shaft; and
a needle guide ramp at the distal end of the elongated shaft and configured to bend the needle as the needle moves distally along the elongated shaft.

16. The apparatus of claim 14, wherein the needle guide system comprises a needle guide ramp in an internal cavity of the applicator.

17. The apparatus of any one of claims 14-16, wherein the elongated shaft is shaped so that the cryotherapy delivery feature is off-axis to a central axis of the apparatus.

18. A method for delivering an anesthetic agent to a target tissue in a nasal cavity of a patient, the method comprising:
inserting a cryotherapy device into the nasal cavity of the patient, wherein the cryotherapy device comprises a cryotherapy delivery feature and one or more protrusions;
positioning the cryotherapy delivery feature in contact with the target tissue;
delivering, using the cryotherapy delivery feature, a cryotherapy treatment to the target tissue;
after inserting the cryotherapy device into the nasal cavity, actuating the one or more protrusions from a retracted state to an extended state;
after actuating the one or more protrusions to the extended state, penetrating the target tissue with the one or more protrusions; and
after penetrating the target tissue, delivering, via the one or more protrusions, an anesthetic agent into the target tissue.

19. The method of claim 18, further comprising:
generating heat in a scaffolding of the cryotherapy device;
transferring the heat from the scaffolding to the one or more protrusions; and
transferring the heat from the one or more protrusions to the target tissue.

20. The method of claim 19, wherein generating the heat in the scaffolding comprises transducing an electrical current via resistive heating.
